(19) United States
(12) Patent Application Publication
(10) Pub. No.: US 2021/0145989 A1
(43) Pub. Date: May 20, 2021

(54) COMPOSITIONS AND RELATED METHODS FOR BLOCKING OFF-TARGET LOCALIZATION OF MANNOSYLATED DEXTRANS AND OTHER CD206 LIGANDS

(71) Applicant: Navidea Biopharmaceuticals, Inc., Dublin, OH (US)

(72) Inventors: David A. Ralph, Columbus, OH (US); Jeffery Arnold, Andover, MA (US)

(21) Appl. No.: 17/039,409

(22) Filed: Sep. 30, 2020

Related U.S. Application Data
(60) Provisional application No. 62/908,136, filed on Sep. 30, 2019.

(51) Int. Cl. A61K 51/06; A61K 51/04; A61P 35/00

(52) U.S. Cl. CPC A61K 51/065; A61P 35/00; A61K 51/0491; A61K 51/0495

(57) ABSTRACT

Disclosed is a method for increase target specificity of a mannosylated dextran therapeutic or diagnostic compound by administering at least a blocking composition comprising a backbone and one or more CD206 targeting moieties attached thereto; administering an effective amount of the mannosylated dextran therapeutic or diagnostic compound comprising a dextran backbone and one or more CD206 targeting moieties and one or more therapeutic agents attached thereto. In exemplary implementations, the molecular mass of the blocking composition backbone is at least about two times larger than the molecular mass of the mannosylated dextran backbone compound.

### CROSS-REFERENCE TO RELATED APPLICATION(S)

This application claims the benefit under 35 U.S.C. § 119(e) to U.S. Provisional Application 62/908,136, filed Sep. 30, 2019, and entitled “Compositions and Related Methods for Blocking Off-Target Localization of Mannosylated Dextrans and Other Cd206 Ligands,” which is hereby incorporated herein by reference in its entirety for all purposes.

### BACKGROUND OF THE INVENTION

Macrophages are significant contributors to the pathobiology of many societally significant illnesses. In lesions of macrophage involved illnesses, macrophages may occur in large numbers. Examples of macrophage involved illnesses include but are not limited to cancer, atherosclerosis, and rheumatoid arthritis. Macrophages respond to various stimuli in their local microenvironment by altering their expression patterns for many genes, potentially hundreds. Such phenotypically altered macrophages are said to be activated macrophages. Depending upon to which stimuli a macrophage is responding, a wide range of activated phenotypic states can be attained. Among those genes that can be differentially expressed upon macrophage activation is the macrophage mannose receptor, CD206, which is highly up regulated in a large proportion of activated macrophages residing at sites of inflammation such as but not limited to tumors (i.e. tumor associated macrophages—TAMs), atherosclerosis, and rheumatoid arthritis.

Because macrophages are both abundant in the lesions of macrophage involved illnesses and contribute significantly to the pathobiology of these diseases, many investigators have targeted imaging agents to macrophages as a tool to diagnose these conditions (examples include) and/or have targeted therapeutics to macrophages as a strategy to treat macrophage involved conditions (examples include). Also, because CD206 is typically highly expressed on activated macrophages residing in lesions of macrophage involved illnesses, many investigators have evaluated CD206 targeted agents to either image and/or treat these lesions or illnesses.

CD206 is a transmembrane C-type lectin with high affinity for binding ligands displaying multiple moieties of the sugar, mannose. CD206 is expressed on various cell types of the myeloid lineage including macrophages and myeloid derived suppressor cells, and some populations of dendritic cells and microglia. Importantly, CD206 is expressed by Kupffer cells. Kupffer cells are resident macrophage-like cells of the liver that occur in large numbers along the walls of sinusoid capillaries. Thus, Kupffer cells are in direct contact with the blood. Another important class of CD206 expressing cell is comprised of the mesangial cells, which reside in the glomeruli of the kidneys. Mesangial cells are separated from the blood only by endothelial cells that do not have an associated basement membrane, thus affording mesangial cells largely unobstructed contact with macromolecules in the blood.

Tilmanocept is a synthetic molecular construct created by attaching mannose moieties and the chelating agent, diethylenetriamine pentaacetic acid (DTPA) to a 10 kD dextran backbone via amine terminated leashes. Tilmanocept has an average of 17 mannose moieties, 5 DTPA moieties, and various numbers of unoccupied amine terminated leashes per dextran backbone molecule. Tilmanocept is a member of a class of related constructs referred to as mannosylated dextrans. Mannosylated dextrans can be constructed using dextrans of varying sizes with varying numbers of mannose moieties attached to the dextran backbone by molecular leashes of varying compositions. To these constructs, numerous types of other moieties, DTPA being but one example, may be conjugated. Mannosylated dextrans generally and tilmanocept specifically are intentionally designed to be high affinity ligands for certain C-type lectins and especially for the macrophage mannose receptor, CD206. However, off-target localization, especially through binding to CD206 expressing cells in the liver and kidney, has undesirable and/or dose limiting consequences. Accordingly, there is need in the art for a method to increasing the target specificity of mannosylated dextran-based therapeutics and diagnostics.

### BRIEF SUMMARY OF THE INVENTION

Disclosed is a method for increase target specificity of a mannosylated dextran therapeutic or diagnostic compound by administering at least a blocking composition comprising a backbone and one or more CD206 targeting moieties attached thereto; administering an effective amount of the mannosylated dextran therapeutic or diagnostic compound comprising a dextran backbone and one or more CD206 targeting moieties and one or more therapeutic agents attached thereto. In exemplary implementations, the molecular mass of the blocking composition backbone is at least two times larger than the molecular mass of the mannosylated dextran backbone compound.

In certain aspects, the mannosylated dextran therapeutic or diagnostic compound is a compound of Formula (I):

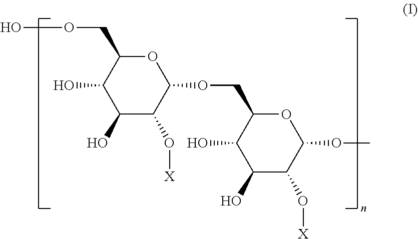

wherein
each X is independently H, L1-A, or L2-R;
each L1 and L2 are independently linkers;
each A independently comprises a therapeutic agent, a diagnostic agent, or H;
each R independently comprises a mannose-binding C-type lectin receptor targeting moiety or H;
and n is an integer greater than zero; and
wherein at least one R comprises a mannose-binding C-type lectin receptor targeting moiety selected from the group consisting of mannose, fucose, and n-acetylglucosamine and at least one A comprises a therapeutic agent or diagnostic agent.

In certain aspects, the blocking compound backbone is chosen from dextran, cellulose, polyethylene glycol, or a polypeptide. In exemplary implementations, the backbone is a dextran.

In further aspects, the one or more CD206 targeting moieties is attached to the blocking compound backbone with a leash.

According to certain aspects, the blocking compound backbone is at least about 35 kDa. In further aspects, the blocking compound backbone is from about 35 kDa and to 180 kDa. In yet further aspects, the blocking compound backbone is from about 35 kDa to 500 kDa.

According to certain aspects, the blocking compound is a compound of Formula (I):

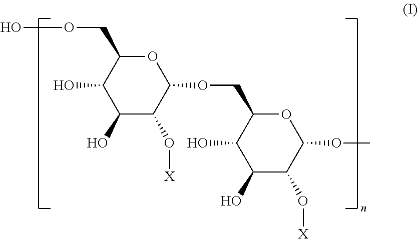

wherein
each X is H;
each R independently comprises a mannose-binding C-type lectin receptor targeting moiety or H;
and n is an integer greater than zero; and
wherein at least one R comprises a mannose-binding C-type lectin receptor targeting moiety selected from the group consisting of mannose, fucose, and n-acetylglucosamine.

In certain aspects, the blocking compound backbone is about 110 kDa and the mannosylated dextran therapeutic or diagnostic compound dextran backbone is about 10 kDa.

In certain aspects, the blocking compound does not contain a therapeutic or diagnostic agent.

In certain aspects, the step of administering the blocking compound is followed by a time interval of from about 1 second to 60 minutes before the step of administering the mannosylated dextran therapeutic or diagnostic compound. In further aspects, the time interval is from about ten minutes to about twenty minutes. In certain alternative embodiments, the blocking compound and the mannosylated dextran therapeutic or diagnostic compound are administered simultaneously.

According to certain aspects, the mannosylated dextran therapeutic or diagnostic compound comprises at least one therapeutic moiety.

In certain aspects, the portion of the injected dose of the mannosylated dextran therapeutic or diagnostic compound that localizes to a desired target tissue other than the liver, kidney, and/or spleen is higher than the localizing portion of the mannosylated dextran therapeutic or diagnostic compound without administration of the blocking compound. In further aspects, the effective dose of the mannosylated dextran therapeutic or diagnostic compound is lower than the effective does of the mannosylated dextran therapeutic or diagnostic compound without administration of the blocking compound. In yet further aspects, the blocking compound preferentially binds to CD206 expressing cells in the liver, kidney, and/or spleen. In further aspects, the mannosylated dextran therapeutic or diagnostic compound has decreased binding to CD206 cells in the liver, kidney, and/or spleen relative to a subject administered a comparable dose of mannosylated dextran therapeutic or diagnostic compound without administration of the blocking compound.

In certain aspects, the subject has been diagnosed with an autoimmune disease, an inflammatory disease, or cancer.

Further disclosed herein is a kit for the diagnosis or treatment of a subject in need thereof including a blocking compound comprising a backbone and one or more CD206 targeting moieties attached thereto; mannosylated dextran therapeutic or diagnostic compound comprising a dextran backbone and one or more CD206 targeting moieties and one or more therapeutic agents attached thereto; and where the molecular mass of the blocking composition backbone is at least two times larger than the molecular mass of the mannosylated dextran backbone compound. In certain aspects, the kit includes a mannosylated dextran therapeutic or diagnostic compound is a compound of Formula (I):

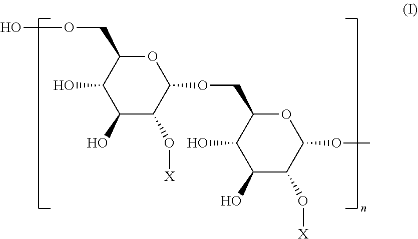

wherein
each X is independently H, L1-A, or L2-R;
each L1 and L2 are independently linkers;
each A independently comprises a therapeutic agent, a diagnostic agent, or H;
each R independently comprises a mannose-binding C-type lectin receptor targeting moiety or H;
and n is an integer greater than zero; and
wherein at least one R comprises a mannose-binding C-type lectin receptor targeting moiety selected from the group consisting of mannose, fucose, and n-acetylglucosamine and at least one A comprises a therapeutic agent or diagnostic agent. In further aspects, the blocking compound backbone is about 110 kDa and the mannosylated dextran therapeutic or diagnostic compound dextran backbone is about 10 kDa.

### BRIEF DESCRIPTION OF THE FIGURES

FIG. 1 shows an autoradiograph of a section from a male Sprague Dawley rat inject IV (tail vein) one hour previously with 25 μg of 99mTc-tilmanocept labeled with 5 mCi of 99mtechnetium showing intense localization to the liver (L) and kidney (K), according to certain embodiments.

FIG. 2A shows an exemplary false color florescence images of Balb/C mice with 4T1 tumors injected IV with Cy5-tilmanocept (A).

FIG. 2B shows the liver (L), kidney (K), spleen (S) and tumor (T) dissected from the mouse shown in FIG. 2A. Note that the false color scales in FIG. 2A and FIG. 2B are different and that the red areas of FIG. 2B have the same level of fluorescence as the yellow areas in FIG. 2A.

FIG. 2C shows an exemplary false color florescence images of Balb/C mice with 4T1 tumors without injection with Cy5-tilmanocept (C).

FIG. 3 shows a PET scan of a normal subject injected with 68-gallium showing extensive localization to the spleen, liver and kidneys. Localization to the bladder is due to urine excretion.

FIG. 4A shows 90 min dynamic PET+CT. L; Liver, K; Kidney, S; Spleen. Blocking Agent Reduces Liver Localization; in this example Wistar rats injected with 5 μg of [⁶⁸Ga]DOTA-Tilmanocept labeled with 300 μCi [⁶⁸Ga] (300 μL) via tail vein catheter. Blocking agent was a 350 kD mannosylated dextran administered immediately prior (650 μL) to [⁶⁸Ga]DOTA-Tilmanocept.

FIG. 4B shows 90 min dynamic PET+CT. L; Liver, K; Kidney, S; Spleen. Blocking Agent Reduces Liver Localization; in this example Wistar rats injected with 5 μg of [⁶⁸Ga]DOTA-Tilmanocept labeled with 300 μCi [⁶⁸Ga] (300 μL) via tail vein catheter. Blocking agent was a 350 kD mannosylated dextran administered immediately prior (650 μL) to [⁶⁸Ga]DOTA-Tilmanocept.

FIG. 5 shows liver localization of [⁶⁸Ga]DOTA-Tilmanocept visualized and quantified by PET-CT in Wistar rats. All rats received intravenously (IV) administration of 5 μg of [⁶⁸Ga]DOTA-Tilmanocept labeled with 300 μCi [⁶⁸Ga]. One rat did not receive the blacking agent. 3 rats were injected IV with 2.5 mg of a 350 kD blocking agent immediately prior to administration of [⁶⁸Ga]DOTA-Tilmanocept.

FIG. 6 shows muscle localization of [⁶⁸Ga]DOTA-Tilmanocept visualized and quantified by PET-CT in Wistar rats. All rats received intravenously (IV) administration of 5 μg of [⁶⁸Ga]DOTA-Tilmanocept labeled with 300 μCi [⁶⁸Ga]. One rat did not receive the blacking agent. 3 rats were injected IV with 2.5 mg of a 350 kD blocking agent immediately prior to administration of [⁶⁸Ga]DOTA-Tilmanocept.

FIG. 7 shows the ratio of Muscle/Live Localization (×100) of [⁶⁸Ga] DOTA-Tilmanocept (% ID/gram) in Blacked and Non-Blocked Rats. In rats injected with [⁶⁸Ga]DOTA-Tilmanocept with and without blocking agent. The ratio of [⁶⁸Ga]DOTA-Tilmanocept localization of muscle/liver (×100) shows that decreasing liver localization through use of the high molecular weight blocking agent is accompanied by increased relative localization to deep tissue macrophages in muscle. Increased relative localization ranged from 2.7× to over 8×.

### DETAILED DESCRIPTION

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH2CH2O— units in the polyester, regardless of whether ethylene glycol was used to prepare the polyester. Similarly, a sebacic acid residue in a polyester refers to one or more —CO(CH2)8CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A1,” “A2,” “A3,” and “A4” are used herein as generic symbols to represent various specific substituents. These symbols can be any substituent, not limited to those disclosed herein, and when they are defined to be certain substituents in one instance, they can, in another instance, be defined as some other substituents.

“R1,” “R2,” “R3,” “Rn,” where n is an integer, as used herein can, independently, possess one or more of the groups listed above. For example, if R1 is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, the term “pharmaceutically acceptable carrier” or “carrier” refers to sterile aqueous or nonaqueous solutions, colloids, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, cancer of the small intestine, and cancer of the esophagus. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. In some embodiments, the present methods can be used to treat a subject having an epithelial cancer, e.g., a solid tumor of epithelial origin, e.g., lung, breast, ovarian, prostate, renal, pancreatic, or colon cancer.

As used herein, the term “subject” refers to the target of administration, e.g., an animal. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more cancer disorders prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with cancer” means having been subjected to a physical examination by a person of skill, for example, a physician, and found to have a condition that can be diagnosed or treated by a compound or composition that can reduce tumor size or slow rate of tumor growth. A subject having cancer, tumor, or at least one cancer or tumor cell, may be identified using methods known in the art. For example, the anatomical position, gross size, and/or cellular composition of cancer cells or a tumor may be determined using contrast-enhanced MRI or CT. Additional methods for identifying cancer cells can include, but are not limited to, ultrasound, bone scan, surgical biopsy, and biological markers (e.g., serum protein levels and gene expression profiles). An imaging solution comprising a cell-sensitizing composition of the present invention may be used in combination with MRI or CT, for example, to identify cancer cells.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, administration to specific organs through invasion, intramuscular administration, intratumoral administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Effective dosages may be estimated initially from in vitro assays. For example, an initial dosage for use in animals may be formulated to achieve a circulating blood or serum concentration of active compound that is at or above an IC50 of the particular compound as measured in an in vitro assay. Calculating dosages to achieve such circulating blood or serum concentrations, taking into account the bioavailability of the particular active agent, is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, which is hereby incorporated by reference in its entirety, and the references cited therein.

The phrase “anti-cancer composition” can include compositions that exert antineoplastic, chemotherapeutic, antiviral, antimitotic, antitumorgenic, anti-angiogenic, anti-metastatic and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agents available in commercial use, in clinical evaluation and in pre-clinical development, which could be included in this application by combination drug chemotherapy. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

“Tilmanocept” refers to a non-radiolabeled precursor of the LYMPHOSEEK® diagnostic agent. Tilmanocept is a mannosylaminodextran. It has a dextran backbone to which a plurality of amino-terminated leashes (—O(CH₂)₃S(CH₂)₂NH₂) are attached to the core glucose elements. In addition, mannose moieties are conjugated to amino groups of a number of the leashes, and the chelator diethylenetriamine pentaacetic acid (DTPA) may be conjugated to the amino group of other leashes not containing the mannose. Tilmanocept generally, has a dextran backbone, in which a plurality of the glucose residues comprise an amino-terminated leash:

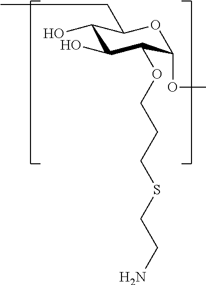

the mannose moieties are conjugated to the amino groups of the leash via an amidine linker:

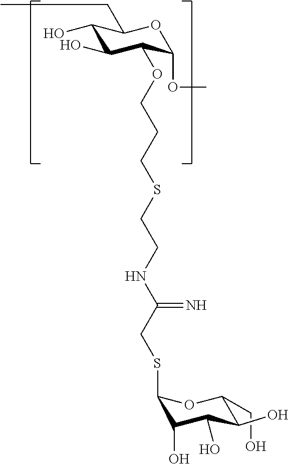

the chelator diethylenetriamine pentaacetic acid (DTPA) is conjugated to the amino groups of the leash via an amide linker:

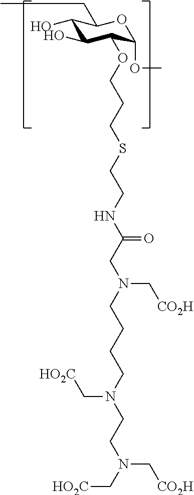

Tilmanocept has the chemical name dextran 3-[(2-aminoethyl)thio]propyl 17-carboxy-10,13,16-tris(carboxymethyl)-8-oxo-4-thia-7,10,13,16-tetraazaheptadec-1-yl 3-[[2-[[1-imino-2-(D-mannopyranosylthio)ethyl]amino]ethyl]thio]propyl ether complexes, and tilmanocept Tc99m has the following molecular formula: [C₆H₁₀O₅]ₙ.(C₁₉H₂₈N₄O₉S^{99m}Tc)_{b}.(C₁₃H₂₄N₂O₅S₂)_{c}.(C₅H₁₁NS)ₐ and contains 3-8 conjugated DTPA molecules (b); 12-20 conjugated mannose molecules (c); and 0-17 amine side chains (a) remaining free. Tilmanocept has the following general structure:

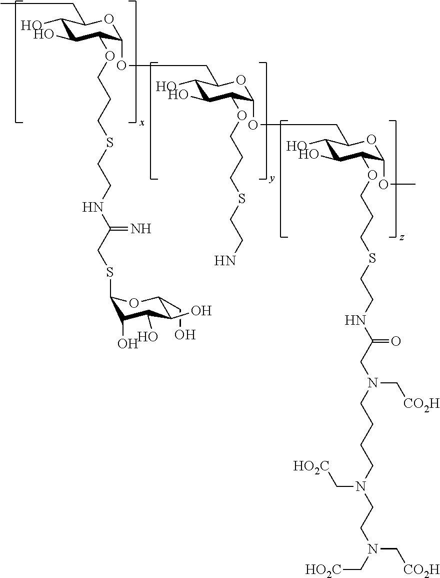

Certain of the glucose moieties may have no attached amino-terminated leash.

This instant disclosure describes compositions of matter and methods for their use of high molecular mannosylated dextrans for blocking (i.e. excluding through competition) the ability of relatively smaller mannosylated dextrans carrying diagnostic and therapeutic moieties to bind to CD206 receptor expressing cells in the liver and kidneys without proportionally diminishing the abilities of these smaller mannosylated dextrans to bind to CD206 cells that have aggregated at sites of pathological processes. The utility of the disclosed invention is that it enables robust localization of mannosylated dextrans carrying diagnostic and therapeutic moieties to sites of pathological processes while reducing or eliminating localization to off-target sites such as the liver and kidneys. Off-target localization has undesirable and/or dose limiting consequences. Also, other diagnostic imaging and therapeutic agents bind to receptors that occur in the liver, kidneys and/or other off-target sites as well as sites of pathological processes. Similar to the situation with mannosylated dextrans, high molecular weight dextrans can be conjugated to these other diagnostic imaging and/or therapeutic agents either directly or through molecular leashes of varying compositions and used to block accumulation of these other diagnostic imaging and/or therapeutic agents in off-target sites while permitting localization of their unconjugated (smaller molecular weight) forms to pathological lesions.

In certain aspects, disclosed is a method for increase target specificity of a mannosylated dextran therapeutic or diagnostic compound by administering at least a blocking composition comprising a backbone and one or more CD206 targeting moieties attached thereto; administering an effective amount of the mannosylated dextran therapeutic or diagnostic compound comprising a dextran backbone and one or more CD206 targeting moieties and one or more therapeutic agents attached thereto. In exemplary implementations, the molecular mass of the blocking composition backbone is at least two times larger than the molecular mass of the mannosylated dextran backbone compound.

In certain aspects, the disclosed method increases specificity for a target tissue in which the CD206 expressing cells do not have or have less extensive direct contact with circulating blood, such as is the case with Kupffer cells of the liver (e.g., the target tissue is not bathed in blood). Such targets may include joints (e.g. for the diagnosis of rheumatoid arthritis) and various cancers outside of the liver and kidneys. The ability to increase specificity for muscle will be appreciated by those skilled in the art as proxy for the ability to increase target specificity in other tissues with limited vascularization.

### Mannosylated Dextran Therapeutic or Diagnostic Compounds

In certain aspects, compounds disclosed herein employ a carrier construct comprising a polymeric (e.g. carbohydrate) backbone having conjugated thereto mannose-binding C-lectin type receptor targeting moieties (e.g. mannose) to deliver one or more active therapeutic agent. Examples of such constructs include mannosylamino dextrans (MAD), which comprise a dextran backbone having mannose molecules conjugated to glucose residues of the backbone and having an active pharmaceutical ingredient conjugated to glucose residues of the backbone. Tilmanocept is a specific example of an MAD. A tilmanocept derivative that is tilmanocept without DTPA conjugated thereto is a further example of an MAD.

In certain implementations, the disclosure provides a compound comprising a dextran-based moiety or backbone having one or more mannose-binding C-type lectin receptor targeting moieties and one or more therapeutic agents attached thereto. The dextran-based moiety generally comprises a dextran backbone similar to that described in U.S. Pat. No. 6,409,990 (the '990 patent), which is incorporated herein by reference. Thus, the backbone comprises a plurality of glucose moieties (i.e., residues) primarily linked by α-1,6 glycosidic bonds. Other linkages such as α-1,4 and/or α-1,3 bonds may also be present. In some embodiments, not every backbone moiety is substituted. In some embodiments, mannose-binding C-type lectin receptor targeting moieties are attached to between about 10% and about 50% of the glucose residues of the dextran backbone, or between about 20% and about 45% of the glucose residues, or between about 25% and about 40% of the glucose residues. In some embodiments, the dextran backbone has a MW of between about 1 and about 20 kDa, while in other embodiments the dextran backbone has a MW of between about 5 and about 15 kDa. In still other embodiments, the dextran backbone has a MW of between about 8 and about 15 kDa, such as about 10 kDa. While in other embodiments the dextran backbone has a MW of between about 1 and about 5 kDa, such as about 2 kDa.

According to further aspects, the mannose-binding C-type lectin receptor targeting moiety is selected from, but not limited to, mannose, fucose, and n-acetylglucosamine. In some embodiments, the targeting moieties are attached to between about 10% and about 50% of the glucose residues of the dextran backbone, or between about 20% and about 45% of the glucose residues, or between about 25% and about 40% of the glucose residues. MWs referenced herein, as well as the number and degree of conjugation of receptor substrates, leashes, and diagnostic/therapeutic moieties attached to the dextran backbone refer to average amounts for a given quantity of carrier molecules, since the synthesis techniques will result in some variability.

According to certain embodiments, the one or more mannose-binding C-type lectin receptor targeting moieties and one or more therapeutic or diagnostic agents are attached to the dextran-based moiety by way of a linker. The linker may be attached at from about 50% to about 100% of the backbone moieties or about 70% to about 90%. The linkers may be the same or different. In some embodiments, the linker is an amino-terminated linker. In some embodiments, the linkers may comprise —O(CH2)3S(CH2)2NH—. In some embodiments, the linker may be a chain of from 1 to 20 member atoms selected from carbon, oxygen, sulfur, nitrogen and phosphorus. The linker may be a straight chain or branched. The linker may also be substituted with one or more substituents including, but not limited to, halo groups, perfluoroalkyl groups, perfluoroalkoxy groups, alkyl groups, such C1-4 alkyl, alkenyl groups, such as C1-4 alkenyl, alkynyl groups, such as C1-4 alkynyl, hydroxy groups, oxo groups, mercapto groups, alkylthio groups, alkoxy groups, nitro groups, azidealkyl groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl- and dialkylamino groups, carbamoyl groups, alkylcarbonyl groups, alkylcarbonyloxy groups, alkoxycarbonyl groups, alkylaminocarbonyl groups, dialkylamino carbonyl groups, arylcarbonyl groups, aryloxycarbonyl groups, alkylsulfonyl groups, arylsulfonyl groups, —NH—NH2; ═N—H; ═N— alkyl; —SH; —S-alkyl; —NH—C(O)—; —NH—C(═N)— and the like. As would be apparent to one skilled in the art, other suitable linkers are possible.

In some embodiments, the one or more therapeutic agent is attached via a biodegradable linker. In some embodiments, the biodegradable linker comprises a pH sensitive moiety, such as a hydrazone. At lower (more acidic) pH, hydrazone linkers spontaneously hydrolyze at increasing rates as pH decreases. When a mannosylated dextran binds to CD206, it is internalized to endosomes which become increasingly acidified over time, thereby releasing the therapeutic agent payloads intracellularly.

According to further embodiments, the therapeutic agent is a cytotoxic agent (e.g. doxorubicin). In still further embodiments, the therapeutic agent is an anti-cancer agent.

In certain aspects, a chelating agent may be attached to or incorporated into a disclosed compound, and used to chelate a therapeutic agent, such as Cu(II). Exemplary chelators include but are not limited to DTPA (such as Mx-DTPA), DOTA, TETA, NETA or NOTA. According to certain exemplary implementations, the chelator is DOTA.

Any of a variety of detectable moieties can be attached to the carrier molecule, directly or indirectly, for a variety of purposes. As used herein, the term “detectable moiety” or “diagnostic moiety” (which these terms may be used interchangeably) means an atom, isotope, or chemical structure which is: (1) capable of attachment to the carrier molecule; (2) non-toxic to humans; and (3) provides a directly or indirectly detectable signal, particularly a signal which not only can be measured but whose intensity is related (e.g., proportional) to the amount of the detectable moiety. The signal may be detected by any suitable means, including spectroscopic, electrical, optical, magnetic, auditory, radio signal, or palpation detection means as well as by the measurement processes described herein.

Suitable detectable moieties include, but are not limited to radioisotopes (radionuclides), fluorophores, chemiluminescent agents, bioluminescent agents, magnetic moieties (including paramagnetic moieties), metals (e.g., for use as contrast agents), RFID moieties, enzymatic reactants, colorimetric release agents, dyes, and particulate-forming agents.

By way of specific example, suitable diagnostic moieties include, but are not limited to:
contrast agents suitable for magnetic resonance imaging (MRI), such as gadolinium (Gd3+), paramagnetic and superparamagnetic materials such as superparamagnetic iron oxide;
contrast agents suitable for computed tomographic (CT) imaging, such as iodinated molecules, ytterbium and dysprosium;
radioisotopes suitable for scintigraphic imaging (or scintigraphy) such as 99mTc, 210Bi, 212-n-213-n-214n.131n Ba, 140n Ba, 11 C,-, 14\hat{ }C, 51, C-.r, 67\hat{ }da, 68\hat{ }a, 153\hat{ }d, 88v Y, 90v Y, 91v Y, 123T 124T, Bi, Bi, Bi, 1, 1, 125I, 131I, i nIn, 115mIn, 18F, 13N, 105Rh, 153Sm, 67Cu, “Cu, 166Ho, 177Lu, 223Ra, 62Rb, 186Re and 188Re, 32P, 33P, 46Sc, 47Sc, 72Se, 75Se, 35S, 89Sr, 182Ta, 123mTe, 127Te, 129Te, 132Te, 65Zn and 89Zr, 95Zr; or other chelateable isotope(s);
gamma-emitting agents suitable for single-photon emission computed tomography (SPECT), such as 99mTc, mIn, and 123I.
dyes and fluorescent agents suitable for optical imaging
agents suitable for positron emission tomography (PET) such as 18F.

A diagnostic moiety can be attached to the carrier molecule in a variety of ways, such as by direct attachment or using a chelator attached to a carrier molecule. In some embodiments, diagnostic moieties can be attached using leashes attached to a carrier backbone. Thereafter, and as described in the ties as by direct attack can be conjugated to an amino group of one or more leashes and can be used to bind the diagnostic moiety thereto. It should be noted that in some instances, glucose moieties may have no attached aminothiol leash. Certain embodiments may include a single type of diagnostic moiety or a mixture of different diagnostic moieties. For example, an embodiment of a compound disclosed herein may comprise a contrast agent suitable for MRI and a radioisotope suitable for scintigraphic imaging, and further combinations of the diagnostic moieties described herein.

One or more diagnostic moieties can be attached to the one or more leashes using a suitable chelator. Suitable chelators include ones known to those skilled in the art or hereafter developed, such as, for example but without limitation, tetraazacyclododecanetetraacetic acid (DOTA), mercaptoacetylglycylglycyl-glycine (MAG3), diethylenetriamine pentaacetic acid (DTP A), dimercaptosuccinic acid, diphenylehtylene diamine, porphyrin, iminodiacetic acid, and ethylenediaminetetraacetic acid (EDTA).

In certain aspects, the disclosed compounds are present in the form of a pharmaceutically acceptable carrier.

According to certain embodiments, the disclose compound is a compound of Formula (I):

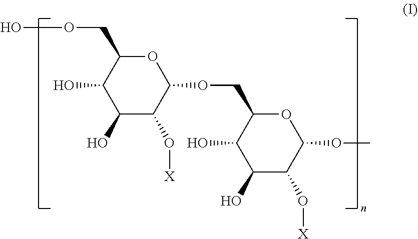

wherein each X is independently H, L1-A, or L2-R;
each L1 and L2 are independently linkers;
each A independently comprises a therapeutic agent or H;
each R independently comprises a mannose-binding C-type lectin receptor targeting moiety or H;
and n is an integer greater than zero; and
wherein at least one R comprises a mannose-binding C-type lectin receptor targeting moiety selected from the group consisting of mannose, fucose, and n-acetylglucosamine and at least one A comprises a therapeutic agent.

In certain embodiments, at least one L1 comprises —(CH2)pS(CH2)-NH—, wherein p and q are integers from 0 to 5.

According to further embodiments, at least one L2 is a C2-12 hydrocarbon chain optionally interrupted by up to three heteroatoms selected from the group consisting of O, S and N.

In still further embodiments, at least one L2 comprises —(CH2)pS(CH2)-NH—, wherein p and q independently are integers from 0 to 5.

In further embodiments, the disclosed composition is of formula (II)

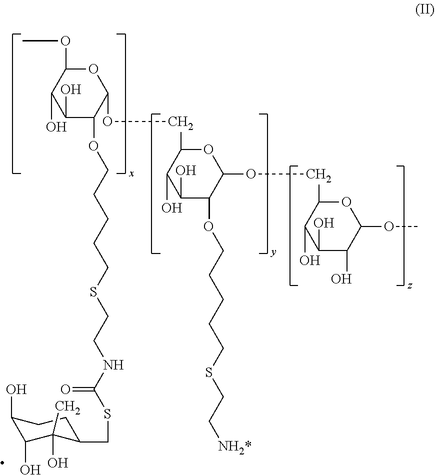

wherein the * indicates the point at which the therapeutic or diagnostic agent is attached. In certain embodiments, the therapeutic agent is attached via a linker.

According to certain embodiments, the disclosed compounds (e.g., the mannosylated dextran therapeutic or diagnostic compound and/or the blocking compound) can include a pharmaceutically acceptable carrier and a compound or a pharmaceutically acceptable salt of the compounds disclosed herein. The disclosed compounds, or pharmaceutically acceptable salts thereof, can also be included in pharmaceutical compositions in combination with one or more other therapeutically active compounds.

### Blocking Compounds

In certain aspects, blocking compounds disclosed herein employ a carrier construct comprising a polymeric (e.g. carbohydrate) backbone having conjugated thereto mannose-binding C-lectin type receptor targeting moieties (e.g. mannose) to preferentially bind to CD206 expressed in the kidney and/or liver. Examples of such constructs include mannosylamino dextrans (MAD), which comprise a dextran backbone having mannose molecules conjugated to glucose residues of the backbone. In alternative embodiments, the blocking composition backbone may be comprised of any polymer suitable for safe administration to subject and the conjugation of C-lectin type receptor targeting moieties (with or without a leash). Examples include, but are not limited to cellulose, polyethylene glycol, and various polypeptides.

In certain aspect, the blocking compound backbone is about 35-500 kD. The blocking compound backbone may be at least about 50 kD, at least about 60 kD, at least about 70 kD, at least about 80 kD, at least about 90 kD, at least about 100 kD, at least about 110 kD, at least about 120 kD, at least about 130 kD, at least about 140 kD, at least about 150 kD, at least about 150 kD, at least about 160 kD, at least about 170 kD, at least about 180 kD, at least about 190 kD, at least about 200 kD, at least about 210 kD, at least about 220 kD, at least about 230 kD, at least about 240 kD, and at least about 250 kD. The blocking compound backbone may be less than about 100 kD, less than about 90 kD, less than about 80 kD, less than about 70 kD, or less than about 60 kD.

According to certain embodiments, the blocking compound backbone has a molecular mass from about 1.5 to about 50 times greater than that of the mannosylated dextran therapeutic or diagnostic compound backbone. In certain aspects, the blocking compound backbone has a molecular mass from about 2 to about 3 times greater than that of the mannosylated dextran therapeutic or diagnostic compound backbone. In further embodiments, the blocking compound backbone has a molecular mass about 2 times greater than that of the mannosylated dextran therapeutic or diagnostic compound backbone.

In some embodiments, the mannose-binding C-type lectin receptor targeting moiety is selected from, but not limited to, mannose, fucose, and n-acetylglucosamine. In some embodiments, the targeting moieties are attached to between about 10% and about 50% of the available residues of the blocking compound backbone, or between about 20% and about 45% of the residues, or between about 25% and about 40% of the residues.

According to certain embodiments, the one or more mannose-binding C-type lectin receptor targeting moieties are attached to the backbone by way of a linker. The linker may be attached at from about 50% to about 100% of the backbone moieties or about 70% to about 90%. The linkers may be the same or different. In some embodiments, the linker is an amino-terminated linker. In some embodiments, the linkers may comprise —O(CH2)3S(CH2)2NH—. In some embodiments, the linker may be a chain of from 1 to 20 member atoms selected from carbon, oxygen, sulfur, nitrogen and phosphorus. The linker may be a straight chain or branched. The linker may also be substituted with one or more substituents including, but not limited to, halo groups, perfluoroalkyl groups, perfluoroalkoxy groups, alkyl groups, such C1-4 alkyl, alkenyl groups, such as C1-4 alkenyl, alkynyl groups, such as C1-4 alkynyl, hydroxy groups, oxo groups, mercapto groups, alkylthio groups, alkoxy groups, nitro groups, azidealkyl groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl- and dialkylamino groups, carbamoyl groups, alkylcarbonyl groups, alkylcarbonyloxy groups, alkoxycarbonyl groups, alkylaminocarbonyl groups, dialkylamino carbonyl groups, arylcarbonyl groups, aryloxycarbonyl groups, alkylsulfonyl groups, arylsulfonyl groups, —NH—NH2; ═N—H; ═N-alkyl; —SH; —S-alkyl; —NH—C(O)—; —NH—C(═N)— and the like. As would be apparent to one skilled in the art, other suitable linkers are possible. In certain alternative embodiments, the targeting moiety is attached directly to the backbone without use of a linker.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

The method of any preceding claim, wherein the blocking compound does not contain a therapeutic or diagnostic agent.

According to certain embodiments, disclosed is a method for increase target specificity of a mannosylated dextran therapeutic or diagnostic compound by administering at least a blocking composition comprising a backbone and one or more CD206 targeting moieties attached thereto; administering an effective amount of the mannosylated dextran therapeutic or diagnostic compound comprising a dextran backbone and one or more CD206 targeting moieties and one or more therapeutic agents attached thereto. In exemplary implementations, the molecular mass of the blocking composition backbone is at least two times larger than the molecular mass of the mannosylated dextran backbone compound.

In certain aspects, the step of administering the blocking compound is followed by a time interval before the administration of the mannosylated dextran therapeutic or diagnostic compound. According to these embodiments, during this time interval, the block compound circulates throughout the body of the subject and binds CD206 expressing cells in the kidney and liver, to allow for subsequent competitive exclusion of the mannosylated dextran therapeutic or diagnostic compound from binding to such cells. In certain aspects, the time interval is at least about ten minutes. In further aspects, the time interval is between about 10 minutes and about 60 minutes. In further aspects, the time interval is from 10 minutes to about 30 minutes. In still further aspects, the time interval is from 10 minutes to about 20 minutes.

According to certain embodiments of the disclosed method, the a mannosylated dextran therapeutic or diagnostic compound comprises at least one therapeutic moiety. In certain exemplary implementations of these embodiments, the effective dose of the mannosylated dextran therapeutic or diagnostic compound is lower than the effective does of the mannosylated dextran therapeutic or diagnostic compound without administration of the blocking compound.

According to further embodiments, the blocking compound preferentially binds to CD206 expressing cells in the liver and/or kidney. In exemplary implementations, the mannosylated dextran therapeutic or diagnostic compound has decreased binding to CD206 cells in the liver and/or kidney relative to a subject administered a comparable dose of mannosylated dextran therapeutic or diagnostic compound without administration of the blocking compound.

In certain aspects, the compound is administered in a therapeutically effective amount. The compound is administered in prophylactically effective amount.

In yet further aspects, the method further comprises administering the compound(s) intravenously, intraperitoneally, intramuscularly, orally, subcutaneously intraocularly, intra-tumor injection or transdermally or delivered directly to tumor organ by invasive techniques.

The methods provided herein may be practiced in an adjuvant setting. In some embodiments, the method is practiced in a neoadjuvant setting, i.e., the method may be carried out before the primary/definitive therapy. In some embodiments, the method is used to treat an individual who has previously been treated. Any of the methods of treatment provided herein may be used to treat an individual who has not previously been treated. In some embodiments, the method is used as a first line therapy. In some embodiments, the method is used as a second line therapy.

According to certain aspects, the subject has been diagnosed with melanoma, breast cancer, lung carcinoma, pancreatic carcinoma, renal carcinoma, ovarian, prostate or cervical carcinoma, glioblastoma, or colorectal carcinoma, cerebrospinal tumor, head and neck cancer, thymoma, mesothelioma, esophageal cancer, stomach cancer, liver cancer, pancreatic cancer, bile duct cancer, bladder cancer, testicular cancer, germ cell tumor, ovarian cancer, uterine cervical cancer, endometrial cancer, lymphoma, acute leukemia, chronic leukemia, multiple myeloma, sarcoma, or any combination thereof.

In certain aspects, the method further comprises administering the composition as a bolus and/or at regular intervals. In certain aspects, the disclosed method further comprises administering the composition intravenously, intraperitoneally, intramuscularly, orally, subcutaneously, intra-tumorally or transdermally.

According to certain further embodiments, the method further comprises diagnosing the subject with cancer. In further aspects, the subject is diagnosed with cancer prior to administration of the composition. According to still further aspects, the method further comprises evaluating the efficacy of the composition. In yet further aspects, evaluating the efficacy of the composition comprises measuring tumor size prior to administering the composition and measuring tumor size after administering the compound. In even further aspects, evaluating the efficacy of the composition occurs at regular intervals. According to certain aspects, the disclosed method further comprises optionally adjusting at least one aspect of method. In yet further aspects, adjusting at least one aspect of method comprises changing the dose of the composition, the frequency of administration of the composition, or the route of administration of the compound.

According to certain alternative embodiments, the subject has been diagnosed with a disease associated with elevated levels of CD206+ macrophages and/or MDSC. Such diseases or conditions include, but are not limited to: acquired immune deficiency syndrome (AIDS), acute disseminated encephalomyelitis (ADEM), Addison's disease, agammaglobulinemia, allergic diseases, alopecia areata, Alzheimer's disease, amyotrophic lateral sclerosis, ankylosing spondylitis, antiphospholipid syndrome, antisynthetase syndrome, arterial plaque disorder, asthma, atherosclerosis, atopic allergy, atopic dermatitis, autoimmune aplastic anemia, autoimmune cardiomyopathy, autoimmune enteropathy, autoimmune hemolytic anemia, autoimmune hepatitis, autoimmune hypothyroidism, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Balo disease/Balo concentric sclerosis, Behcet's disease, Berger's disease, Bickerstaffs encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, chronic venous stasis ulcers, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Dego's disease, Dercum's disease, dermatitis herpetiformis, dermatomyositis, Diabetes mellitus type I, Diabetes mellitus type II diffuse cutaneous systemic sclerosis, Dressler's syndrome, drug-induced lupus, discoid lupus erythematosus, eczema, emphysema, endometriosis, enthesitis-related arthritis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, Evan's syndrome, fibrodysplasia ossificans progressive, fibrosing alveolitis (or idiopathic pulmonary fibrosis), gastritis, gastrointestinal pemphigoid, Gaucher's disease, glomerulonephritis, Goodpasture's syndrome, Graves' disease, Guillain-Barré syndrome (GBS), Hashimoto's encephalopathy, Hashimoto's thyroiditis, heart disease, Henoch-Schonlein purpura, herpes gestationis (aka gestational pemphigoid), hidradenitis suppurativa, HIV infection, Hughes-Stovin syndrome, hypogammaglobulinemia, infectious diseases (including bacterial infectious diseases), idiopathic inflammatory demyelinating diseases, idiopathic pulmonary fibrosis, idiopathic thrombocytopenic purpura, IgA nephropathy, inclusion body myositis, inflammatory arthritis, inflammatory bowel disease, inflammatory dementia, interstitial cystitis, interstitial pneumonitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosus, linear IgA disease (LAD), lupoid hepatitis (aka autoimmune hepatitis), lupus erythematosus, lymphomatoid granulomatosis, Majeed syndrome, malignancies including cancers (e.g., sarcoma, Kaposi's sarcoma, lymphoma, leukemia, carcinoma and melanoma), Meniere's disease, microscopic polyangiitis, Miller-Fisher syndrome, mixed connective tissue disease, morphea, Mucha-Habermann disease (aka Pityriasis lichenoides et varioliformis acuta), multiple sclerosis, myasthenia gravis, myositis, narcolepsy, neuromyelitis optica (aka Devic's disease), neuromyotonia, occular cicatricial pemphigoid, opsoclonus myoclonus syndrome, Ord's thyroiditis, palindromic rheumatism, PANDAS (pediatric autoimmune neuropsychiatric disorders associated with streptococcus), paraneoplastic cerebellar degeneration, Parkinsonian disorders, paroxysmal nocturnal hemoglobinuria (PNH), Parry Romberg syndrome, Parsonage-Turner syndrome, pars planitis, pemphigus vulgaris, peripheral artery disease, pernicious anaemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restenosis, restless leg syndrome, retroperitoneal fibrosis, rheumatoid arthritis, rheumatic fever, sarcoidosis, schizophrenia, Schmidt syndrome, Schnitzler syndrome, scleritis, scleroderma, sepsis, serum Sickness, Sjögren's syndrome, spondyloarthropathy, Still's disease (adult onset), stiff person syndrome, stroke, subacute bacterial endocarditis (SBE), Susac's syndrome, Sweet's syndrome, Sydenham chorea, sympathetic ophthalmia, systemic lupus erythematosus, Takayasu's arteritis, temporal arteritis (aka “giant cell arteritis”), thrombocytopenia, Tolosa-Hunt syndrome,) transplant (e.g., heart/lung transplants) rejection reactions, transverse myelitis, tuberculosis, ulcerative colitis, undifferentiated connective tissue disease, undifferentiated spondyloarthropathy, urticarial vasculitis, vasculitis, vitiligo, and Wegener's granulomatosis.

Also provided herein are kits of pharmaceutical formulations containing the disclosed compounds or compositions. The kits may be organized to indicate a single formulation or combination of formulations. The composition may be sub-divided to contain appropriate quantities of the compound. The unit dosage can be packaged compositions such as packeted powders, vials, ampoules, prefilled syringes or sachets containing liquids.

The compound or composition described herein may be a single dose or for continuous or periodic discontinuous administration. For continuous administration, a kit may include the compound in each dosage unit. For periodic discontinuation, the kit may include placebos during periods when the compound is not delivered. When varying concentrations of the composition, the components of the composition, or relative ratios of the compound or other agents within a composition over time is desired, a kit may contain a sequence of dosage units.

The kit may contain packaging or a container with the compound formulated for the desired delivery route. The kit may also contain dosing instructions, an insert regarding the compound, instructions for monitoring circulating levels of the compound, or combinations thereof. Materials for performing using the compound may further be included and include, without limitation, reagents, well plates, containers, markers or labels, and the like. Such kits are packaged in a manner suitable for treatment of a desired indication. Other suitable components to include in such kits will be readily apparent to one of skill in the art, taking into consideration the desired indication and the delivery route. The kits also may include, or be packaged with, instruments for assisting with the injection/administration or placement of the compound within the body of the subject. Such instruments include, without limitation, an inhalant, syringe, pipette, forceps, measuring spoon, eye dropper or any such medically approved delivery means. Other instrumentation may include a device that permits reading or monitoring reactions in vitro.

The compound or composition of these kits also may be provided in dried, lyophilized, or liquid forms. When reagents or components are provided as a dried form, reconstitution generally is by the addition of a solvent. The solvent may be provided in another packaging means and may be selected by one skilled in the art.

A number of packages or kits are known to those skilled in the art for dispensing pharmaceutical agents. In one embodiment, the package is a labeled blister package, dial dispenser package, or bottle.

In certain aspects, the kit disclosed herein includes a blocking compound comprising a backbone and one or more CD206 targeting moieties attached thereto; a mannosylated dextran therapeutic or diagnostic compound comprising a dextran backbone and one or more CD206 targeting moieties and one or more therapeutic agents attached thereto; and where the molecular mass of the blocking composition backbone is at least two times larger than the molecular mass of the mannosylated dextran backbone compound. In certain aspects, the kit includes a mannosylated dextran therapeutic or diagnostic compound is a compound of Formula (I):

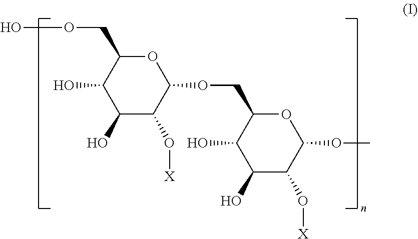

wherein
each X is independently H, L1-A, or L2-R;
each L1 and L2 are independently linkers;
each A independently comprises a therapeutic agent, a diagnostic agent, or H;
each R independently comprises a mannose-binding C-type lectin receptor targeting moiety or H;
and n is an integer greater than zero; and
wherein at least one R comprises a mannose-binding C-type lectin receptor targeting moiety selected from the group consisting of mannose, fucose, and n-acetylglucosamine and at least one A comprises a therapeutic agent or diagnostic agent. In further aspects, the blocking compound backbone is about 110 kDa and the mannosylated dextran therapeutic or diagnostic compound dextran backbone is about 10 kDa.

### EXAMPLES

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1

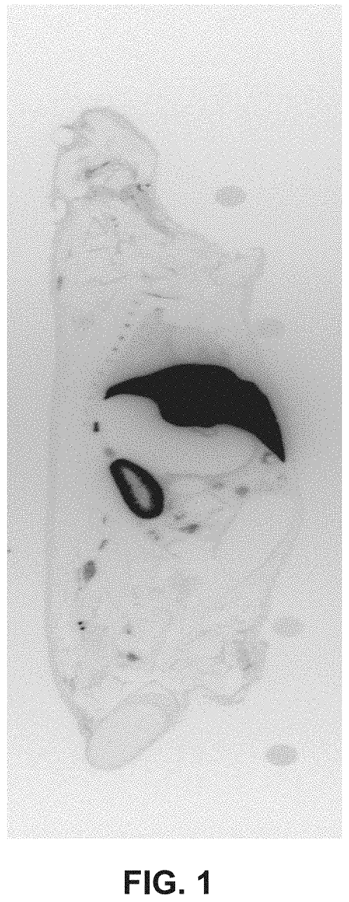

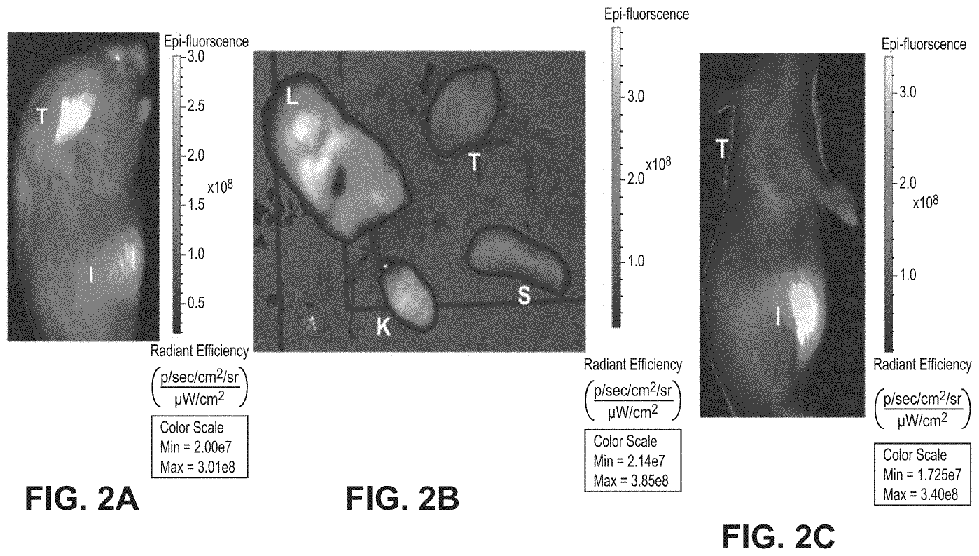

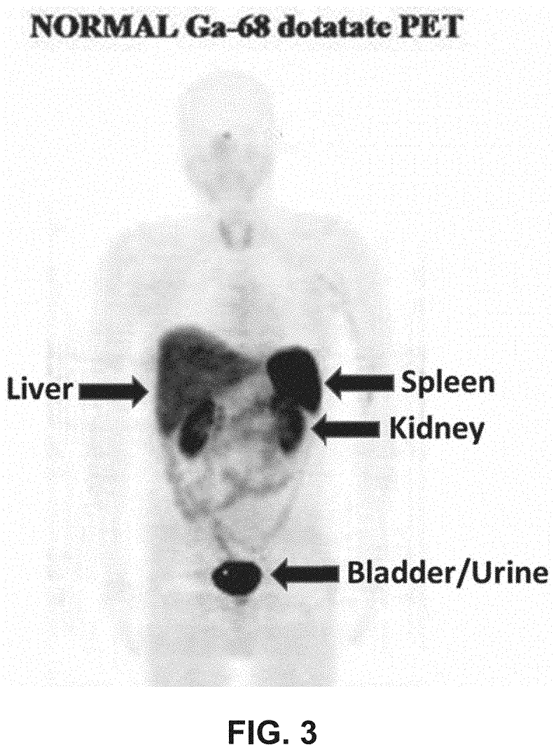

Because of their high affinity for CD206, mannosylated dextrans generally and 99mTc-tilmanocept specifically have been investigated as either imaging agents or drug delivery vehicles for macrophage involved illnesses atherosclerosis, cancer and rheumatoid arthritis (unpublished results). Diagnostic imaging studies were performed using Lymphoseek (99mTc-tilmanocept) that was administered by intravenous (IV) injection rather than the peritumoral or intradermal injection route of administration used for the SLN related indications. The results of these investigations were generally positive; however, as shown in FIGS. 1 and 2, IV administration of 99mTc-tilmanocept resulted in highly significant uptake of the mannosylated dextran by the liver and kidneys. FIG. 1 shows an autoradiogram of a section through a male Sprague Dawley rat inject IV (tail vein) one hour previously with 25 μg of 99mTc-tilmanocept labeled with 5 mCi of ^{99m}technetium. A large portion of the injected radioactivity was excreted into the urine by one hour after injection (not shown in FIG. 1). Among the organs, the greatest localization of radioactivity occurred in the liver and kidneys (L and K respectively in FIG. 1) due to large numbers of Kupffer cells and mesangial cells that reside in these organs. There was also significant (but lesser) localization to the spleen where macrophages are known to reside. The Sprague Dawley rats in this study were healthy, so there were no disease lesions to which 99mTc-tilmanocept could have localized.

### Example 2

In a second experiment, a fluorescently labeled (Cy5) tilmanocept was injected into Balb/C mice with a 4T1 syngeneic tumors. When excited, Cy5 fluoresces in the near infrared region of the light spectrum. FIG. 2 provides false color images from this experiment, in which yellow indicates areas with higher fluorescence than red areas. FIG. 2A shows an image of a mouse injected with the Cy5-tilmanocept, while FIG. 2C shows an image of an animal that was not injected with any fluorescent material. Comparing FIG. 2A with FIG. 2C reveals that there was considerable autofluorescence from the animals' fur and intestines (I). However, localization of the Cy5-tilmanocept in the tumor (T) is clearly evident in the animal injected with the fluorescent mannosylated dextran. The animal shown in FIG. 2A was dissected permitting the fluorescence of the liver, kidney, spleen and tumor to be examined (FIG. 2B). In FIG. 2B it is evident that the tumor (T) specifically localized a significant amount of Cy5-tilmanocept due to CD206 expression on TAMs. The intensity of localizations to the liver (L) and kidney (K) were greater than the tumor due to the presence of CD206 expressing Kupffer cells and mesangial cells respectively. The intensity of fluorescence was similar in the spleen (S) and tumor due to the presence of macrophages in the spleen and TAMs in the tumor. Off-target uptake by the liver and kidneys negatively impacts localization to pathological lesions with large numbers of CD206 expressing macrophages and could result is undesirable off target toxicities for CD206 targeted mannosylated dextran drug delivery constructs.

### Example 3

To block the localization of small molecular weight mannosylated dextrans, such as 99mTc-tilmanocept or similarly sized drug delivery vehicles, to off-target sites such as the liver or kidneys, a high molecular weight mannosylated dextran can be synthesized. Dextrans are polymers of glucose with α-1,6 glycosidic linkages. The mannosylation of dextrans is described in the '990 patent. The synthesis of tilmanocept is described in the '990 patent. In this example, tilmanocept is synthesized beginning with a 10 kDa dextran backbone. The high molecular weight mannosylated dextran can be synthesized beginning with a 110 kDa dextran backbone. To each of these backbones, amine terminated leashes can be added to the dextran backbones as previously described the '990 patent. (In the case of tilmanocept, the chelating agent, DTPA, can then be added to a portion of the amine terminated leashes. For the large molecular weight construct, no chelating agent or any other detection or therapeutic moiety are added. Finally, for both tilmanocept and the high molecular weight construct, various numbers of the sugar, mannose, can be added to a portion of the unoccupied amine terminated leashes. It should be noted that other sugars in addition to mannose can be attached to the amine terminated leashes to create a construct that will bind to CD206; however, attachment of multiple mannose moieties is both necessary and sufficient to enable high affinity binding to CD206.

### Example 4

A High Molecular Weight Mannosylated Dextran Administered Intravenously (IV) Immediately Prior to IV Administration of 68Gallium Labeled DOTA-Tilmanocept Results in Selective Blocking (i.e. Decrease) of Liver Localization and Increased Localization to Deep Tissue Macrophages in Muscle.

A tilmanocept derivative was synthesized that carried the chelator dodecane tetraacetic acid (DOTA) instead of diethylenetriaminepentaacetic acid (DTPA). This construct was termed DOTA-tilmanocept and is identical to the commercially available tilmanocept except that the DTPA on commercial tilmanocept was exchanged for DOTA. This exchange of DTPA for DOTA was performed so that the construct, DOTA-tilmanocept, could be effectively labeled with various ions including ⁶⁸gallium [⁶⁸Ga]. ⁶⁸Gallium enables imaging by positron emission tomography (PET), which was the imaging modality used in this example. DOTA-tilmanocept has an average molecular weight of ≈20 kD.

An experiment was conducted in which 4 Wistar rats (≈250 grams) were injected intravenously (IV) with 5 μg of [⁶⁸Ga] DOTA-tilmanocept labeled with 300 μCi of ⁶⁸gallium. Dynamic PET imaging was conducted from the time of injection to 90 minutes post injection. Standard uptake values (SUV) were calculated to determine the biodistribution of the radiolabel to various organs and reported out as the percent of injected dose per gram (% ID/gr) of each organ. This example reports the result from two organs, the liver and the thigh muscle. The thigh muscle was used as a generalizable model for localization of [⁶⁸Ga] DOTA-tilmanocept to tissue macrophages that are not directly exposed to the blood. Tumor associated macrophages and macrophages associated with rheumatoid arthritis pathobiology are two examples, among many, of tissue macrophages. In contrast, the liver contains large numbers of Kupffer cells that express CD206 and are directly exposed to the blood circulation. Kupffer cells represent a significant sink for mannosylated dextrans that are injected into the blood.

A high molecular weight (HMW) mannosylated dextran (Mw≈350 kD) was synthesized using the same chemical procedure used to create tilmanocept or DOTA-tilmanocept except that the starting dextran backbone was 150 kD instead of 10 kD and that no chelator was conjugated to the resulting mannosylated dextran. This HMW construct was designed to bind to CD206 on Kupffer cells, thereby blocking localization of [⁶⁸Ga] DOTA-tilmanocept to the liver, but also to exit the blood flow and penetrate inefficiently into tissues due to its relatively large size. The relatively poor penetration into tissues would prevent the HMW construct from competing with [⁶⁸Ga] DOTA-tilmanocept for localization to tissue macrophages. In addition, because less [⁶⁸Ga] DOTA-tilmanocept would be localizing to the Kupffer cells, more [⁶⁸Ga] DOTA-tilmanocept would be available to exit the blood circulation and bind to CD206 expressed on tissue macrophages.

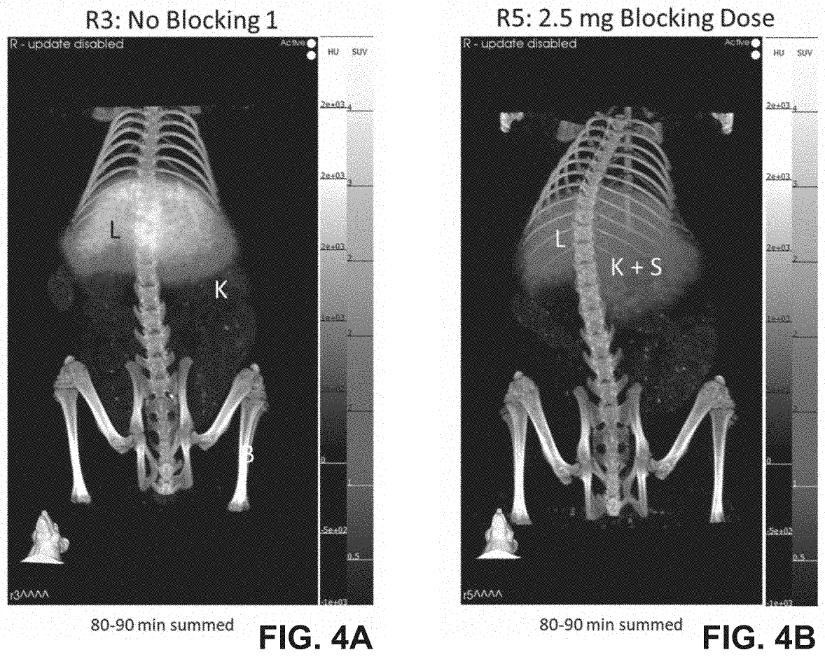

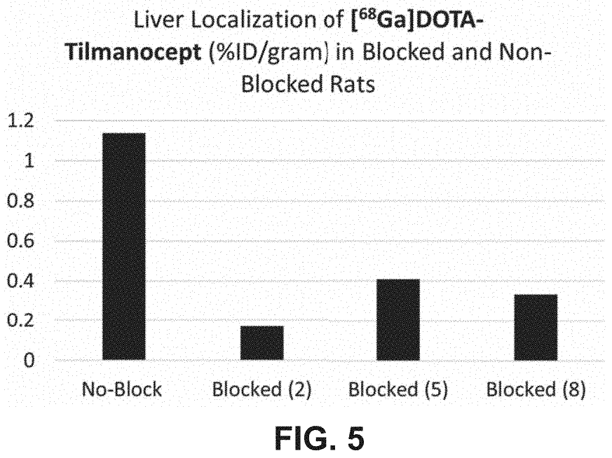

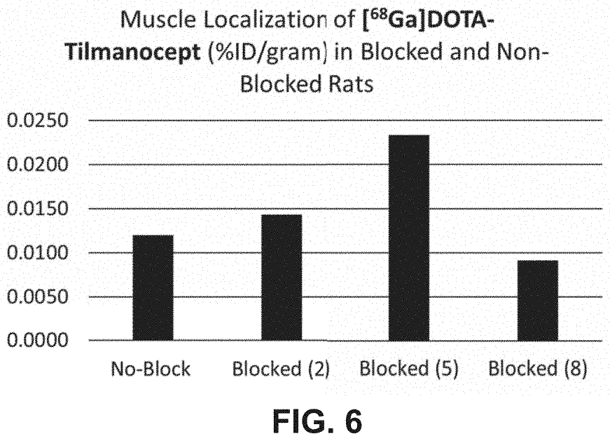

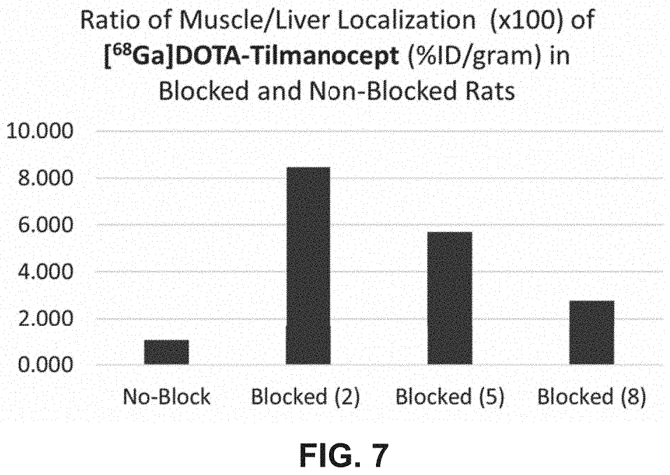

In the experiment described in this example, 3 of 4 rats were injected IV with 2.5 mg of the HMW blocking construct immediately prior to IV administration of [⁶⁸Ga] DOTA-tilmanocept. In FIGS. 4A and 4B, representative 80-90 minute PET-CT images of the rat not administered the HMW blocking agent (FIG. 4A) and a rat administered the HWM blocking agent (FIG. 4B) are shown. Qualitative evaluation of the images shows that the HMW blocking agent reduced [⁶⁸Ga] DOTA-tilmanocept localization to the liver. FIG. 5 shows a graph of localization of [⁶⁸Ga] DOTA-tilmanocept to the livers expressed as % ID/gr of the non-blocked rat and the 3 rats administered the HMW blocking agent. On average, the rats that were administered the HMW blocking agent had 26.7% as much localization as the non-blocked rat. FIG. 6 shows the amount of localization of [⁶⁸Ga] DOTA-tilmanocept expressed as % ID/gr in the thigh muscles of the 4 rats. While results varied, on average the rats administered the HMW blocking agent had 29.7% greater localization than was observed in the rat that had not received the blocking agent. In a third graphical representation of the results (FIG. 7), the ratio of the % ID/gr of the thigh muscle/the liver (×100) is shown for each of the 4 rats. All rats administered the HMW blocking agent had higher muscle/liver ratios, ranging from 2.7× to 8.5× greater than was observed in the rat not administered the blocking agent. These results show that the HMW mannosylated dextran blocking agent selectively inhibited the binding of [⁶⁸Ga] DOTA-tilmanocept to the liver but did not decrease and possibly increased the localization of [⁶⁸Ga] DOTA-tilmanocept to macrophages in the thigh muscle. Other sites of tissue macrophages, such as sites of macrophage involved pathological lesions, are expected to perform similarly.

Although the present invention has been described with reference to preferred embodiments, persons skilled in the art will recognize that changes may be made in form and detail without departing from the spirit and scope of the invention.

What is claimed is:

1. A method for increase target specificity of a mannosylated dextran therapeutic or diagnostic compound comprising:
a. administering at least a blocking compound comprising a backbone and one or more CD206 targeting moieties attached thereto;
b. administering an effective amount of the mannosylated dextran therapeutic or diagnostic compound comprising a dextran backbone and one or more CD206 targeting moieties and one or more therapeutic or diagnostic agents attached thereto; and
wherein the molecular mass of the blocking composition backbone is at least two times larger than the molecular mass of the mannosylated dextran backbone compound.

2. The method of claim 1, wherein the mannosylated dextran therapeutic or diagnostic compound is a compound of Formula (I): 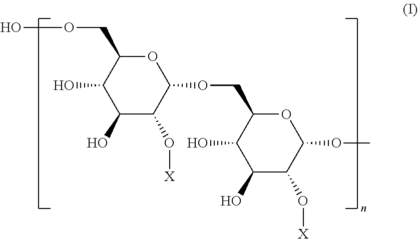
wherein
each X is independently H, L1-A, or L2-R;
each L1 and L2 are independently linkers;
each A independently comprises a therapeutic agent, a diagnostic agent, or H;
each R independently comprises a mannose-binding C-type lectin receptor targeting moiety or H;
and n is an integer greater than zero; and
wherein at least one R comprises a mannose-binding C-type lectin receptor targeting moiety selected from the group consisting of mannose, fucose, and n-acetylglucosamine and at least one A comprises a therapeutic agent or diagnostic agent.

3. The method of claim 1 wherein the blocking compound backbone is selected from a list consisting of: dextran, cellulose, polyethylene glycol, and polypeptides.

4. The method of claim 3, wherein the backbone is a dextran.

5. The method of claim 1, wherein the one or more CD206 targeting moieties is attached to the blocking compound backbone with a leash.

6. The method of claim 1, wherein the blocking compound backbone is at least about 35 kDa.

7. The method of claim 6, wherein the blocking compound backbone is between about 35 kDa and about 180 kDa.

8. The method of claim 6, wherein the blocking compound backbone is between about 35 kDa and about 500 kDa.

9. The method of claim 6 wherein the blocking compound is a compound of Formula (I): 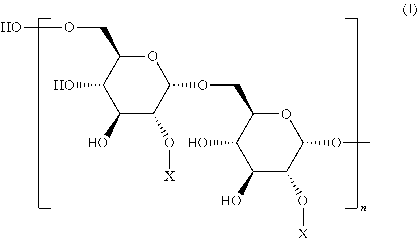
wherein
each X is H;
each R independently comprises a mannose-binding C-type lectin receptor targeting moiety or H;
and n is an integer greater than zero; and
wherein at least one R comprises a mannose-binding C-type lectin receptor targeting moiety selected from the group consisting of mannose, fucose, and n-acetylglucosamine.

10. The method of claim 6, wherein the blocking compound backbone is about 110 kDa and the mannosylated dextran therapeutic or diagnostic compound dextran backbone is about 10 kDa.

11. The method of claim 6, wherein the blocking compound does not contain a therapeutic or diagnostic agent.

12. The method of claim 1, wherein the step of administering the blocking compound is followed by a time interval of at least zero to 60 minutes before the step of administering the mannosylated dextran therapeutic or diagnostic compound.

13. The method of claim 12, wherein the time interval is from about ten minutes to about twenty minutes.

14. The method of claim 1, wherein the blocking compound and the mannosylated dextran therapeutic or diagnostic compound are administered simultaneously.

15. The method of claim 1, wherein the mannosylated dextran therapeutic or diagnostic compound comprises at least one therapeutic moiety.

16. The method of claim 15, wherein the portion of the injected dose of the mannosylated dextran therapeutic or diagnostic compound that localizes to a desired target tissue other than the liver, kidney, and/or spleen is higher than the localizing portion of the mannosylated dextran therapeutic or diagnostic compound without administration of the blocking compound.

17. The method of claim 15, wherein the effective dose of the mannosylated dextran therapeutic or diagnostic compound is lower than the effective does of the mannosylated dextran therapeutic or diagnostic compound without administration of the blocking compound.

18. The method of claim 1, wherein the blocking compound preferentially binds to CD206 expressing cells in the liver, kidney, and/or spleen.

19. The method of claim 18, wherein the mannosylated dextran therapeutic or diagnostic compound has decreased binding to CD206 cells in the liver, kidney, and/or spleen relative to a subject administered a comparable dose of mannosylated dextran therapeutic or diagnostic compound without administration of the blocking compound.

20. The method of claim 1, wherein the subject has been diagnosed with an autoimmune disease, an inflammatory disease, or cancer.

21. A method for increase target specificity of a mannosylated dextran therapeutic or diagnostic compound comprising:
a. administering at least a blocking compound comprising a backbone and one or more CD206 targeting moieties attached thereto wherein the blocking compound is from about 35 kDa to about 500 kDa;
b. administering an effective amount of the mannosylated dextran therapeutic or diagnostic compound comprising a dextran backbone and one or more CD206 targeting moieties and one or more therapeutic or diagnostic agents attached thereto; and
wherein the molecular mass of the blocking composition backbone is at least two times larger than the molecular mass of the mannosylated dextran backbone compound.

22. A kit for the diagnosis or treatment of a subject in need thereof comprising:
a. a blocking compound comprising a backbone and one or more CD206 targeting moieties attached thereto;
b. mannosylated dextran therapeutic or diagnostic compound comprising a dextran backbone and one or more CD206 targeting moieties and one or more therapeutic agents attached thereto; and
wherein the molecular mass of the blocking composition backbone is at least two times larger than the molecular mass of the mannosylated dextran backbone compound.

23. The kit of claim 22, wherein the mannosylated dextran therapeutic or diagnostic compound is a compound of Formula (I): 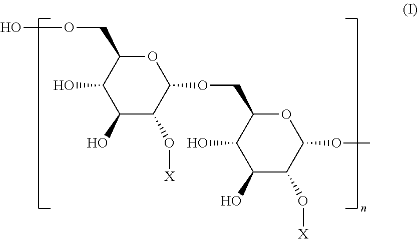
wherein
each X is independently H, L1-A, or L2-R;
each L1 and L2 are independently linkers;
each A independently comprises a therapeutic agent, a diagnostic agent, or H;
each R independently comprises a mannose-binding C-type lectin receptor targeting moiety or H;
and n is an integer greater than zero; and
wherein at least one R comprises a mannose-binding C-type lectin receptor targeting moiety selected from the group consisting of mannose, fucose, and n-acetylglucosamine and at least one A comprises a therapeutic agent or diagnostic agent.

24. The kit of claim 22, wherein the blocking compound backbone is about 110 kDa and the mannosylated dextran therapeutic or diagnostic compound dextran backbone is about 10 kDa.
